(19) United States
(12) Patent Application Publication
(10) Pub. No.: US 2020/0190151 A1
(43) Pub. Date: Jun. 18, 2020

(54) KV1.3-BLOCKING VENOM PEPTIDE VARIANTS AND RELATED USES

(71) Applicant: The Scripps Research Institute, La Jolla, CA (US)

(72) Inventors: Richard A. Lerner, La Jolla, CA (US); Hongkai Zhang, San Diego, CA (US)

(21) Appl. No.: 16/099,361

(22) PCT Filed: May 11, 2017
(86) PCT No.: PCT/US2017/032102

§ 371 Date: Nov. 6, 2018

Related U.S. Application Data
(60) Provisional application No. 62/336,151, filed on May 13, 2016.

(51) Int. Cl. C07K 14/435; G01N 33/68; A61P 37/06

(52) U.S. Cl. CPC C07K 14/43522; A61K 38/00; A61P 37/06; G01N 33/6872

(57) ABSTRACT

The invention provides variant venom peptides and polypeptides that specifically block potassium voltage gated ion channel Kv1.3. The invention also provides methods of inhibiting Kv1.3 channel activity, methods for treating disorders that are associated with undesired or aberrant Kv1.3 channel activity, methods for detecting Kv1.3 expression in target cells such as lymphocytes, and methods for diagnosing diseases or disorders that are associated with aberrant Kv1.3 overexpression.

### CROSS-REFERENCE TO RELATED APPLICATIONS

The subject patent application claims the benefit of priority to U.S. Provisional Patent Application Nos. 62/336,151 (filed May 13, 2016). The full disclosure of the priority application is incorporated herein by reference in its entirety and for all purposes.

### BACKGROUND OF THE INVENTION

Ion channels regulate a diversity of cellular functions through generation of ionic currents, including cardiac, CNS, and immune physiology. Kv1.3, the potassium voltage-gated channel subfamily A member 3, is expressed on T cells and functions to regulate T cell activation. Blocking Kv1.3 depolarizes T cells and inhibits calcium entry, cytokine production, and proliferation of activated T cells in vitro. Kv1.3 blockers have been shown to reduce T cell dependent disease progression in autoimmune models, such as experimental autoimmune encephalomyelitis (EAE), experimental arthritis, delayed-type hypersensitivity (DTH), allergic contact dermatitis and glomerulonephritis. Kv1.3 also plays a role in regulating weight gain and improving insulin sensitivity. Kv1.3 blockers have been shown to increase glucose transporter 4 (GLUT4) cell surface expressions in skeletal muscle and adipose tissue, resulting in increased insulin sensitivity in normal and ob/ob obese mice, and to increase glucose uptake in primary adipocytes in vitro. Kv1.3 may have a critical function in smooth muscle proliferative disorders like restenosis in patients following vascular surgery, such as angioplasty. Kv1.3 blockers inhibit calcium entry, reduce smooth muscle cell migration, and inhibit neointimal hyperplasia in ex vivo human vein samples. Kv1.3 channels are also involved in the activation and/or proliferation of many types of cells, including tumor cells, microglia and differentiation of neuronal progenitor cells, suggesting that Kv1.3 blockers may be beneficial in the treatment of neuroinflammatory and neurodegenerative diseases, and cancers.

Toxin peptides produced by a variety of organisms have evolved to target ion channels. They typically bind to the channel pore and physically block the ion conduction pathway or antagonize channel function by binding to a region outside the pore (e.g., the voltage sensor domain). For example, ShK is a known venom peptide that can block Kv1.3 and other related ion channels. Although venom peptide toxins are naturally pre-optimized to be highly potent compounds, their pharmacological and pharmacokinetic properties still need to be optimized.

A need exists in the art for better and more effective Kv1.3 blockers that are useful in treating diseases or disorders that are mediated by Kv1.3 ion channel, e.g., inflammatory and autoimmune diseases such as lupus and multiple sclerosis. The instant invention addresses these and other currently unmet needs in the art.

### SUMMARY OF THE INVENTION

In one aspect, the invention provides isolated or recombinant polypeptides that contain a variant venom peptide. Typically, the variant venom peptide consists of (1) at least the first 18 N-terminal residues of SEQ ID NO:4 or conservatively modified variants thereof, (2) SEQ ID NO:6 except for at least one amino acid substitution selected from the group consisting of I4V, T6M, and I7V, or (3) at least 31 contiguous residues that are at least 90% identical to SEQ ID NO:4. In preferred embodiments, the polypeptides of the invention can specifically bind to and block the Kv1.3 ion channel, e.g., human Kv1.3. In some of the Kv1.3-blocking polypeptides of the invention, the variant venom peptide consists of at least the first 20, 22, 24, 26, 28, 30, 32 or 34 N-terminal residues of SEQ ID NO:4. In some of the Kv1.3-blocking polypeptides, the variant venom peptide consists of SEQ ID NO:6 except for at least two amino acid substitutions selected from the group consisting of I4V, T6M, and I7V. In some of the Kv1.3-blocking polypeptides, the variant venom peptide consists of 35 contiguous residues that are at least 90%, 95% or 98% identical to SEQ ID NO:4. In some Kv1.3-blocking polypeptides of the invention, the variant venom peptide consists of an amino acid sequence as shown in SEQ ID NO:4.

Some Kv1.3-blocking polypeptides of the invention further contain an Fc domain of an IgG that is fused at the N-terminus or C-terminus of the variant venom peptide. In some of these polypeptides, the Fc domain has an amino acid sequence shown in SEQ ID NO:2. In some Kv1.3-blocking polypeptides of the invention, the Fc domain is fused via a linker sequence. In some of these polypeptides, the linker sequence has an amino acid sequence shown in SEQ ID NO:3. In some polypeptides, the Fc domain is fused at the N-terminus via the linker sequence. Some of Kv1.3-blocking polypeptides of the invention contain an amino acid sequence that is at least 90% identical to SEQ ID NO:1. Some of these polypeptides contain an amino acid sequence shown in SEQ ID NO: 1.

In a related aspect, the invention provides isolated or purified polynucleotides that encode the novel Kv1.3-blocking polypeptides described herein. In some related embodiments, the invention provides expression vectors containing one or more of the polynucleotides, as well as host cells harboring such polynucleotides or expression vectors.

In another aspect, the invention provides methods for blocking or suppressing Kv1.3 ion channel activity in a target cell. The methods involve contacting the cell with a therapeutically effective amount of a novel Kv1.3-blocking polypeptide described herein, thereby blocking or suppressing Kv1.3 ion channel activity in the target cell. In some of these methods, the target cell is a lymphocyte such as T cell. In some methods, the target cell is present in vivo in a subject. Some methods of the invention are directed to inhibiting Kv1.3 ion channel activity in a target cell (e.g., T cell) in a subject suffering from a disease or disorder that is associated with or mediated by undesired T cell activation. For example, the subject can be one who is afflicted with an inflammatory disorder or an autoimmune disease.

In another related aspect, the invention provides methods for treating a subject afflicted with or at risk of developing a disease or disorder that is associated with or mediated by undesired or aberrant Kv1.3 ion channel activity or overexpression. The methods entail administering to the subject a pharmaceutical composition containing a therapeutically effective amount of a novel Kv1.3-blocking polypeptide described herein. Some of these methods are directed to treating diseases or disorders that are associated with undesired T cell activation, e.g., inflammatory disorders or autoimmune diseases.

In another aspect, the invention provides methods for detecting expression or overexpression of Kv1.3 ion channel in a target cell. These methods involve (a) contacting the cell with a novel Kv1.3-binding agent described herein (a variant venom peptide or Kv1.3-blocking polypeptide), (b) detecting a signal indicative of a specific binding of the agent to the target cell, and (c) comparing the detected signal to a baseline signal from binding of the agent to a control cell. A substantive departure of the detected signal from the baseline signal indicates overexpression of Kv1.3 in the target cell. In some of these methods, the Kv1.3-binding agent is labeled with a fluorophore, and the specific binding of the agent to the cell is detected by flow cytometry. In some methods, the target cell is isolated from a biological sample from a subject suspected of having a disorder associated with overexpressed Kv1.3. In some of these methods, the target cell is T lymphocyte, and the disorder is an autoimmune disease.

A further understanding of the nature and advantages of the present invention may be realized by reference to the remaining portions of the specification and claims.

### DESCRIPTION OF THE DRAWINGS

FIG. 1 is a schematic illustration of identification of venom peptide and ion channel interaction using the proximity based assay. Membrane tethered venom peptide is coupled to the TEV protease on its cytoplasmic side. Ion channel is fused to a TEV substrate sequence and transcription factor GAL4-VP16 on its cytoplasmic side. Interaction of the ion channel and ligand approximates the TEV and its substrate sequence, resulting in release of GAL4-VP16 for expression of the reporter gene.

FIG. 2 is a schematic illustration of mining the repertoire of natural venom peptides for ion channel drug discovery. (a) Construction of natural venom peptide lentiviral library. Cysteine knot peptides are extracted from the venom database using bioinformatics. The corresponding oligonucleotides are synthesized using high throughput DNA synthesis. Toxin genes are amplified from these oligonucleotides and cloned into lentivirus transfer vector pLigand to produce lentivirus library. (b) Autocrine-based selection from the venom peptide lentiviral library using the proximity based assay. The lentivirus libraries are prepared from lentiviral plasmids and Kv1.3 reporter cells are transduced with the lentiviral library. Interaction between Kv1.3 binding venom peptides and Kv1.3 channel approximates TEV protease and its substrate sequence, resulting in release of transcription factor and expression of tdTomato. Fluorescent cells are sorted and venom peptide-encoding genes are amplified from the sorted cells to make the enriched lentiviral library for the next round of selection. After iterative rounds of selection, enriched peptide sequences are analyzed by NGS sequencing technology.

FIG. 3 shows characterization of the selected peptide CllTx1. Kv1.3 expressing cells were stained by recombinant CllTx1-Fc fusion protein and immunostained cells were analyzed by flow cytometry; authentic CllTx1 peptide isolated from scorpion venom potently blocked Kv1.3 current in patch clamp studies.

FIG. 4 shows correlation between Kv1.3 binding ability and enrichment factor of variants for the selection of combinatorial library. HEK293F cells transiently transfected with Kv1.3 were stained with variants and analyzed with flow cytometry. The fluorescence intensities of the variants were plotted against the enrichment factors calculated from NGS results.

FIG. 5 shows characterization of Fc-ShK variant S1-2. (a) The sequence of variant S1-2 (SEQ ID NO:4). The randomized region of the variant S1-2 is RSCVDMV (SEQ ID NO:13). The linker is GGOGGS (SEQ ID NO:12)×2. (b) IC50 of the variant S1-2 on Kv1.3 current determined by patch clamp electrophysiology. (c) Efficacy of the variant S1-2 on DNFB-induced delayed type hypersensitivity in Lewis rats. Lewis rats were sensitized with DNFB on shaved dorsum on day 0 and challenged with DNFB on both sides of the right pinnae on day 5. Ear thickness changes were measured 24 h after challenge. 16 h and 1 h before DNFB challenge, rats were s.c. dosed with the variant S1-2 and positive control rats were treated with dexamethasone 10 mg/kg p.o. once daily.

FIG. 6 shows 1 proximity based assay vector maps and features. (a) pReceptor vector is used for expression of an ion channel in-frame fusion with an optimized TEV protease substrate sequence and GAL4-VP16 at its C-terminus under control of human Ubiquitin C (UBC) promoter. The entire cassette is flanked by genomic insulator elements for stabilized expression. Expression of neomycin resistance gene (Neo) enables selection of a stable cell line with Geneticin. (b) pLigand Vector is used for ligand expression and based on the 3rd generation of lentiviral transfer vector. UbC promoter is used for low level expression of ligand in-frame fusion with PDGFR-TM and TEV protease at its C-terminus. The murine Interleukin 2 leader sequence targets the ligand construct to secretory pathway. PDGFR-TM anchors the ligand to the plasma membrane and TEV protease cleaves the TEV protease substrate sequence and releases GAL4-VP16 from membrane protein. (c) The reporter Vector contains 7 Upstream Activating Sequences (UAS) for the GAL4 DNA-binding domain upstream of a minimal adenoviral promoter and is designed for transcriptional activation of the reporter genes tdTomato by association of the GAL4-VP16. Expression of puromycin resistance gene (Puro) enables selection of stable cell line by puromycin.

FIG. 7 shows Kv1.3 stable cells were infected with lentivirus encoding venom peptide ShK or Vc1.1. Cells were fixed with paraformaldehyde two days post-infection and cell nuclei were stained with Hoechst 33342. Stained cells were imaged using con-focal microscopy.

FIG. 8 shows selection of Kv1.3 targeting peptide by FACS sorting. Three rounds of selection of the natural venom peptide library(a) and the combinatorial library of Fc-ShK variants(b). The x axis indicates the expression of tdTomato detected by FL2 channel.

### DETAILED DESCRIPTION OF THE INVENTION

### 1. Overview

Animal venoms represent a rich source of pharmacologically active peptides that interact with ion channels. However, a challenge to discovering drugs remains because of the slow pace at which venom peptides are discovered and refined. The present invention is predicated in part on the identification by the present inventors of variant venom peptides that are highly selective in blocking the Kv1.3 ion channel over the other ion channels (see, e.g., Table 3 herein). The inventors utilized an autocrine-based selection format to identify or improve active venoms from a combinatorial library of venom peptide derivatives. As detailed herein, the inventors first developed the efficient autocrine-based high throughput selection system, which was validated by discovering novel Kv1.3 channel blockers from a natural venom peptide library that was formatted for autocrine based selection. The inventors also engineered a library of derivative peptides from sea anemone derived Kv1.3 blocker peptide (ShK). The ShK derivative peptides were then selected to identify Kv1.3-specific blockers with long half-life in vivo.

In accordance with these studies, the invention provides various venom peptide variants or derivatives that are capable of specifically and selectively binding to and blocking the Kv1.3 ion channel. The invention also provides therapeutic applications for treating subjects with conditions or disorders that are mediated by or associated with undesired (overexpressed) or aberrant Kv1.3 expression or ion channel activity, e.g., autoimmune diseases. Further provided in the invention are diagnostic methods of using the Kv1.3-binding agents of the invention to identify subjects who are afflicted with or at risk of developing diseases that are associated with or mediated by aberrant or overexpressed Kv1.3 expression.

A further understanding of the nature and advantages of the present invention may be realized by reference to the remaining portions of the specification and the claims.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The singular terms “a,” “an,” and “the” include plural referents unless the context clearly indicates otherwise. Similarly, the word “or” is intended to include “and” unless the context clearly indicates otherwise.

As used herein, the term “amino acid” of a peptide refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoscrine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The Kv1.3 blocking peptides or fusions thereof of the invention encompass derivative or analogs which have been modified with non-naturally coding amino acids.

The term “contacting” has its normal meaning and refers to combining two or more agents (e.g., polypeptides or polynucleotides), combining agents and cells, or combining two populations of different cells. Contacting can occur in vitro, e.g., mixing two polypeptides or mixing a population of antibodies with a population of cells in a test tube or growth medium. Contacting can also occur in a cell or in situ, e.g., applying a polypeptide agent to a cell, or mixing two polypeptides in a cell by coexpression in the cell of recombinant polynucleotides encoding the two polypeptides, or in a cell lysate.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As used herein, a “derivative” of a reference molecule (e.g., a Kv1.3 blocking peptide disclosed herein) is a molecule that is chemically modified relative to the reference molecule while retaining some or all of the biological activity. The modification can be, e.g., oligomerization or polymerization, modifications of amino acid residues or peptide backbone, cross-linking, cyclization, conjugation, fusion to additional heterologous amino acid sequences, or other modifications that substantially alter the stability, solubility, or other properties of the peptide.

The term “engineered cell” or “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A “fusion” protein or polypeptide refers to a polypeptide comprised of at least two polypeptides and a linking sequence or a linkage to operatively link the two polypeptides into one continuous polypeptide. The two polypeptides linked in a fusion polypeptide are typically derived from two independent sources, and therefore a fusion polypeptide comprises two linked polypeptides not normally found linked in nature.

“Linkage” refers to means of operably or functionally connecting two biomolecules (e.g., polypeptides or polynucleotides encoding two polypeptides), including, without limitation, recombinant fusion, covalent bonding, disulfide bonding ionic bonding, hydrogen bonding, and electrostatic bonding. “Fused” refers to linkage by covalent bonding. A “linker” or “spacer” refers to a molecule or group of molecules that connects two biomolecules, and serves to place the two molecules in a preferred configuration with minimal steric hindrance.

The term “isolated” means a molecule is removed from its natural surroundings. However, some of the components found with it may continue to be with an “isolated” protein. Thus, an “isolated polypeptide” is not as it appears in nature but may be substantially less than 100% pure protein.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, Wis.); or by manual alignment and visual inspection (see, e.g., Brent at al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Kv1.3” (also known as KCNA3, HPCN3, HGK5, HuKIII, or HLK3) refers to the well-known human potassium voltage-gated channel subfamily A member 3 having a sequence shown in UniProt accession number P22001.

Unless otherwise specified, the terms “polypeptide” and “peptide” are used interchangeably herein to refer to a polymer of amino acid residues. They encompass both short oligopeptides (e.g., peptides with less than about 25 residues) and longer polypeptide molecules (e.g., polymers of more than about 25 or 30 amino acid residues). Typically, the Kv1.3 blocking peptides or polypeptides of the invention can comprise from about 5 amino acid residues to about 350 or more amino acid residues in length. In some embodiments, the peptides or polypeptides comprise from about 10 amino acid residues to about 60 amino acid residues in length. In some other embodiments, the agonists can comprise from about 15 amino acid residues to about 40 amino acid residues in length. The Kv1.3 blocking peptides or fusions thereof of the invention include naturally occurring amino acid polymers and non-naturally occurring amino acid polymer, as well as amino acid polymers in which one or more amino acid residue is an artificial chemical mimetic of a corresponding naturally occurring amino acid. Unless otherwise indicated, a particular polypeptide sequence also implicitly encompasses conservatively modified variants thereof.

As used herein, the term “peptide mimetic” or “peptidomimetic” refers to a derivative compound of a reference peptide (e.g., a Kv13 blocking variant venom peptide disclosed herein) that biologically mimics the peptide's functions. Typically, the peptidomimetic derivative of a Kv1 0.3 blocking peptide or fusion thereof has at least 50%, at least 75% or at least 90% of the Kv1.3-blocking activity of the reference polypeptide.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

As used herein, the term “treat” or treatment” refers to administration of compounds or agents to prevent or delay the onset of the symptoms, complications, or biochemical indicia of a disease or condition, alleviating the symptoms or arresting or inhibiting further development of the disease or condition, or disorder. Treatment may be prophylactic (to prevent or delay the onset of the condition, or to prevent the manifestation of clinical or subclinical symptoms thereof) or therapeutic suppression or alleviation of symptoms after the manifestation of the condition.

As used herein, the term “variant” refers to a molecule (e.g., a peptide or polypeptide) that contains a sequence that is substantially identical to the sequence of a reference molecule. For example, the reference molecule can be a Kv1.3 blocking peptide or polypeptide disclosed herein or a fusion thereof. The reference molecule can also be a polynucleotide encoding the Kv1.3 blocking agent disclosed herein or a fusion thereof. In some embodiments, the variant can share at least 50%, at least 70%, at least 80%, at least 90, at least 95% or more sequence identity with the reference molecule. In some other embodiments, the variant differs from the reference molecule by having one or more conservative amino acid substitutions. In some other embodiments, a variant of a reference molecule (e.g., a Kv1.3 blocking peptide or fusion thereof) has altered amino acid sequences (e.g., with one or more conservative amino acid substitutions) but substantially retains the biological activity of the reference molecule. Conservative amino acid substitutions are well known to one skilled in the art.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

### III. Novel Variant Venom Peptides or Polypeptides that Block Kv1.3 Ion Channel

The invention provides novel variant venom peptides or polypeptides containing such peptides that specifically and selectively block the potassium voltage-gated channel Kv1.3. These Kv1.3-blocking agents (polypeptides or peptides) of the invention contain a variant venom peptide (or “venom peptide variant”) that is based on specific sequences identified from a combinatorial library of venom peptide derivatives. As detailed herein, the specific sequences were identified via an autocrine selection format, and are capable of specifically blocking the Kv1.3 channel. Examples of the specific peptide sequences selected from the combinatorial library of venom peptide derivatives include RSCVDMVPKSRCTAFKCKHSMKYRLSFCRKTCGTC (SEQ ID NO:4) (“S1-2” venom peptide variant) and LRCFDLLPKSRCTAFKCKHSMKYRLSFCRKTCGTC (SEQ ID NO:7). Typically, the variant venom peptide in the Kv1.3 blocking polypeptides of the invention contains from about 15 amino acid residues to about 40 amino acid residues in length. In some embodiments, the variant venom peptide in the Kv1.3 blocking polypeptides of the invention is composed of at least the first 18 N-terminal residues of the exemplified sequence (e.g., SEQ ID NO:4) or conservatively modified variants thereof. For example, the variant venom peptide can consist of at least 19, 20, 21, 22, 23, 24, 25, 26, 27, 28, 29, 30, 31, 32, 33, or 34 N-terminal residues of SEQ ID NO:4 or conservatively modified variants thereof. In some of these embodiments, the variant venom peptide has a sequence that is substantially identical to the exemplified sequence (e.g., SEQ ID NO:4). In some other embodiments, the variant venom peptide has a sequence as shown in SEQ ID NO:4 except for one or more conservatively modified residues, e.g., at positions 4, 6, and 7.

In some embodiments, the variant venom peptide in the Kv1.3 blocking polypeptides of the invention is composed of the sequence RSCIDTIPKSRCTAFKCKHSMKYRLSFCRKTCGTC (SEQ ID NO:6) except for at least one amino acid substitution selected from the group consisting of I4V, T6M, and I7V. In some of these embodiments, the variant venom peptide is composed of an amino acid sequence as shown in SEQ ID NO:6 except for at least two of the three noted substitutions. In some embodiments, the variant venom peptide contains all three substitutions I4V, T6M, and I7V relative to SEQ ID NO:6. One or more of the Ile4, Thr6, and Ile7 residues in the sequence can also be substituted with conservative variants of Val, Met, and Val, respectively. In addition to one or more substitutions at these three positions, the variant venom peptide in the Kv1.3 blocking polypeptides of the invention can also contain variations or conservative substitutions at one or more of the other positions in SEQ ID NO:6.

In some embodiments, the variant venom peptide in the Kv1.3 blocking polypeptides of the invention is composed of at least 31 contiguous residues that are substantially identical (e.g., at least 90%) identical to SEQ ID NO:4. In various embodiments, the variant venom peptide is composed of at least 32, 33, 34 or 35 contiguous residues that are substantially identical to SEQ ID NO:4. In various embodiments, the sequence of the variant venom peptide is at least 90%, 91%, 93%, 94%, 95%, 96%, 97%, 98, or 99% identical to at least 31, 32, 33, 34 or 35 contiguous residues of SEQ ID NO:4.

In addition to the variant venom peptide, the Kv1.3 blocking polypeptides of the invention can contain other peptide fragments or motifs that can enhance their pharmaceutical or biological properties. Some of these fusion polypeptides are composed of the variant venom peptide described above and a fusion partner. The fusion partner can be any molecule or moiety that can improve or enhance the biological, pharmacokinetic, or pharmacodynamic properties of the variant venom peptide (e.g., SEQ ID NO:4).

In some embodiments, the fusion partner is covalently bonded to the variant venom peptide via a linker sequence. For example, fusion of a protein or polypeptide partner to the variant venom peptide can be achieved via a linker sequence that contains one or more (e.g., 1, 2, 3, 4, 5, 6, 7, 8, 9, 10, 15, 20 or more) tandem repeats of GGGGS (SEQ ID NO:12).

In some embodiments, the variant venom peptide in the Kv1.3 blocking polypeptides can be fused to the Fc domain of an IgG. Fusion with an Fc-domain provides a number of beneficial biological and pharmacological properties. As exemplified herein for the Fc-S1-2 fusion (SEQ ID NO:1), the presence of the Fc domain can markedly increase the plasma half-life of the hybrid molecule, which prolongs therapeutic activity, owing to its interaction with the salvage neonatal Fc-receptor, as well as to the slower renal clearance for larger sized molecules. The attached Fc domain also enables the molecules to interact with Fc-receptors (FcRs) found on immune cells. As the Fc domain folds independently, it can improve the solubility and stability of the fused variant venom peptide both in vitro and in vivo. Further, the Fc region allows for easy cost-effective purification by protein-G/A affinity chromatography during manufacture. In the Kv1.3 blocking polypeptides of the invention, the variant venom peptide can be fused with an Fc domain at either its N-terminus or C-terminus.

Kv1.3 blocking polypeptides containing a variant venom peptide of the invention and a fusion partner such as Fc domain can be readily generated in accordance with standard recombination techniques, routinely practiced protein synthesis methods or the protocols described herein. As exemplification, a fusion Kv1.3 blocking polypeptide containing a variant venom peptide S1-2 (SEQ ID NO:4) fused to an Fc domain at the N-terminus (Fc-S1-2) is disclosed herein. This fusion polypeptide has a sequence of EPKSCDKTHTCPPCPAPELLGGPSVFLFPPKPKDTLMISRTPEVTCVVVDVSHEDPEV KFNWYVDGVEVHNAKTKPREEQYNSTYRVVSVLTVLHQDWLNGKEYKCKVSNK ALPAPIEKTISKAKGQPREPQVYTLPPSRDELTKNQVSLYCLVKGFYPSDIAVEWESN GQPENNYKTTPPVLDSDGSFFLYSKLTVDKSRWQQGNVFSCSVMHEALHNHYTQK SLSLSPGK-GNSOGGGSGGGGS-RSCVDMVPKSRCTAFKCKHSMKYRLSFCRKTCGTC (SEQ ID NO: 1). In this fusion Kv1.3 blocking polypeptide, the Fc sequence (SEQ ID NO:2) is fused to the N-terminus of the S1-2 variant venom peptide (SEQ ID NO:4) via a linker GNSGGGGSGGGGS (SEQ ID NO:3). In some embodiments, a polynucleotide for expression the Fc-containing fusion polypeptide can additionally contain a sequence encoding a signal peptide to facilitate translocation and secretion. For example, the IL-2 signal peptide can be present at the N-terminus of the Fc-containing fusion polypeptide.

Kv1.3 blocking polypeptides of the invention can also include other analogs, peptidomimetic, derivatives or variants that can be generated from one of the exemplified variant venom peptides and retain the activity in selectively blocking the Kv1.3 ion channel. Unless otherwise noted, these peptides, which are derived from the exemplified variant venom peptides or Kv1.3 blocking polypeptides and have similar or improved activity in selectively blocking Kv1.3 channel, are collectively termed “active variants” of the exemplified venom peptides or Kv1.3 blocking polypeptides. Derivative compounds generated from one of the exemplified peptides can be subject to the assays described herein to identify such “active variants”. In some embodiments, the derivative compounds are modified versions of the exemplified peptides which are generated by conservative amino acid substitutions. In some other embodiments, the derivative compounds are variants produced by non-conservative substitutions to the extent that that they substantially retain the activities of those peptides. Activities of the variant polypeptides in blocking Kv1.3 channel can be examined with the methods described herein or standard techniques routinely practiced in the art. See, e.g., Gill et al., Assay Drug Dev Technol. 2007, 5:373-80; and Nguyen et al., Curr. Med. Chem. 17: 2882-2896, 2010.

In some embodiments, the analogs or derivative compounds of an exemplified variant venom peptide or Kv1.3 blocking polypeptide (e.g., SEQ ID NO:1 or SEQ ID NOs:4 and 7) can contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by polypeptide bonds. Similarly, they can also be cyclic polypeptides and other conformationally constrained structures. Methods for modifying a polypeptide to generate analogs and derivatives are well known in the art, e.g., Roberts and Vellaccio, The Peptides: Analysis, Synthesis, Biology, Eds. Gross and Meinhofer, Vol. 5, p. 341, Academic Press, Inc., New York, N.Y. (1983); and Burger's Medicinal Chemistry and Drug Discovery, Ed. Manfred E. Wolff, Ch. 15, pp. 619-620, John Wiley & Sons Inc., New York, N.Y. (1995).

Some other derivative compounds of the exemplified Kv1.3 blocking polypeptides are peptidomimetics. Peptidomimetics based on a Kv1.3 blocking polypeptide or polypeptide (e.g., SEQ ID NO:1 or SEQ ID NOs:4 and 7) substantially retain the activities of the reference polypeptide. They include chemically modified polypeptides, polypeptide-like molecules containing non-naturally occurring amino acids, peptoids and the like, have a structure substantially the same as the reference polypeptides upon which the peptidomimetic is derived (see, for example, Burger's Medicinal Chemistry and Drug Discovery, 1995, supra). For example, the peptidomimetics can have one or more residues chemically derivatized by reaction of a functional side group. In addition to side group derivatizations, a chemical derivative can have one or more backbone modifications including alpha-amino substitutions such as N-methyl, N-ethyl, N-propyl and the like, and alpha-carbonyl substitutions such as thioester, thioamide, guanidino and the like. Typically, a peptidomimetic shows a considerable degree of structural identity when compared to the reference polypeptide and exhibits characteristics which are recognizable or known as being derived from or related to the reference polypeptide. Peptidomimetics include, for example, organic structures which exhibit similar properties such as charge and charge spacing characteristics of the reference polypeptide. Peptidomimetics also can include constrained structures so as to maintain optimal spacing and charge interactions of the amino acid functional groups.

In some embodiments, derivative compounds of the exemplified Kv1.3 blocking polypeptides (e.g., SEQ ID NO: 1 or SEQ ID NO:4) can contain modifications within the sequence, such as, modification by terminal-NH₂ acylation, e.g., acetylation, or thioglycolic acid amidation, by terminal-carboxylamidation, e.g., with ammonia, methylamine, and the like terminal modifications. One can also modify the amino and/or carboxy termini of the polypeptides described herein. Terminal modifications are useful to reduce susceptibility by proteinase digestion and renal clearance, and therefore can serve to prolong half-life of the polypeptides in solution, particularly in biological fluids where proteases may be present. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In some embodiments, the N-terminus is acetylated with acetic acid or acetic anhydride. In some embodiments, the exemplified Kv1.3 blocking polypeptides can be modified by PEGylation with polyethylene glycol (PEG) polymer.

Carboxy terminus modifications include replacing the free acid with a carboxamide group or forming a cyclic lactam at the carboxy terminus to introduce structural constraints. One can also cyclize the peptides described herein, or incorporate a desamino or descarboxy residue at the termini of the peptide, so that there is no terminal amino or carboxyl group, to decrease susceptibility to proteases or to restrict the conformation of the peptide. Methods of circular peptide synthesis are known in the art, for example, in U.S. Patent Application No. 20090035814; and Muralidharan and Muir, Nat. Methods, 3:429-38, 2006. C-terminal functional groups of the peptides described herein include amide, amide lower alkyl, amide di(lower alkyl), lower alkoxy, hydroxy, and carboxy, and the lower ester derivatives thereof, and the pharmaceutically acceptable salts thereof.

The Kv1.3 blocking polypeptide or variant venom peptide of the invention (e.g., SEQ ID NO: or 4) can also serve as structural models for non-peptidic compounds with similar biological activity. There are a variety of techniques available for constructing compounds with the same or similar desired biological activity as the variant venom peptides or Kv1.3 blocking polypeptides exemplified herein, but with more favorable activity with respect to solubility, stability, and susceptibility to hydrolysis and proteolysis. See, e.g., Morgan and Gainor, Ann. Rep. Med. Chem. 24:243-252, 1989. These techniques include, but are not limited to, replacing the peptide backbone with a backbone composed of phosphonates, amidates, carbamates, sulfonamides, secondary amines, and N-methylamino acids.

The Kv1.3 blocking polypeptides or peptides described herein, including variants and derivatives thereof, can be chemically synthesized and purified by standard chemical or biochemical methods that are well known in the art. Some of the methods for generating analog or derivative compounds of the Kv1.3 blocking polypeptides or peptides are described herein. Other methods that may be employed for producing the Kv1.3 blocking peptides or polypeptides of the invention and their derivative compounds, e.g., solid phase peptide synthesis. Commercial peptide synthesizing machines are available for solid phase peptide synthesis. For example, the Advanced Chemtech Model 396 Multiple Peptide Synthesizer and an Applied Biosystems Model 432A Peptide synthesizer are suitable. There are commercial companies that make custom synthetic peptides to order, e.g., Abbiotec, Abgent, AnaSpec Global Peptide Services, LLC., Invitrogen, and rPeptide, LLC. In some embodiments, the Kv1.3 blocking peptides or polypeptides (e.g., SEQ ID NOs:4 and 7, or SEQ TD NO:1) and derivatives thereof are synthesized and purified by molecular methods that are well known in the art. As detailed below, recombinant production of the polypeptides can be performed in a variety of protein expression systems using host cells selected from the group consisting of mammalian cell lines, insect cell lines, yeast, bacteria, and plant cells.

### IV. Polynucleotides and Vectors for Expressing Kv1.3 Blocking Polypeptides

As exemplified herein for the Fc-S1-2 fusion polypeptide (SEQ ID NO: 1), the venom derived, Kv1.3 blocking peptides or polypeptides of the invention are preferably produced via recombinant techniques. In these methods, polynucleotide sequences encoding the Kv1.3 blocking peptides or polypeptides are usually linked to an appropriate promoter in protein expression vectors or viral vectors. The expression constructs can further contain a secretory sequence to assist purification of the peptide from the cell culture medium. As noted above, the host cells to which the vectors are introduced can be any of a variety of expression host cells well known in the art, e.g., bacteria (e.g., E. coli), yeast cell, or mammalian cells. In some embodiments, recombinant vector carrying the polynucleotide encoding a Kv1.3 blocking polypeptide can also be used far further molecular modifications such as site-directed mutagenesis for a Kv1.3 blocking polypeptide and/or to reduce the immunogenic properties of the peptide or improve protein expression in heterologous expression systems. In some related embodiments, the invention provides isolated or substantially purified polynucleotides (DNA or RNA) which encode the Kv1.3 blocking peptides or polypeptides described herein. Expression vectors and engineered host cells harboring the vectors for expressing polynucleotides encoding the polypeptides are also provided in the invention.

A variety of expression vector/host systems are suitable for expressing the peptides or polypeptides of the invention. Examples include, e.g., microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vector (e.g., cauliflower mosaic virus, CaMV; tobacco mosaic virus, TMV) or transformed with bacterial expression vectors (e.g., Ti or pBR322 plasmid); or animal cell systems. The selection of a particular vector depends upon the intended use of the variant venom peptides or polypeptides. For example, the selected vector must be capable of driving expression of the variant venom peptide or polypeptide in the desired cell type, whether that cell type be prokaryotic or eukaryotic. Many vectors contain sequences allowing both prokaryotic vector replication and eukaryotic expression of operably linked gene sequences. Vectors useful for the invention may be autonomously replicating, that is, the vector exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors and in stably transfected cell lines.

Vectors useful for the invention preferably contain sequences operably linked to the variant venom polypeptide coding sequences that permit the transcription and translation of the encoding polynucleotide sequences. Sequences that permit the transcription of the linked variant venom polypeptide encoding sequences include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence. The promoter sequence can be constitutive or inducible. Examples of constitutive viral promoters include the HSV, TK, RSV, SV40 and CMV promoters. Examples of suitable inducible promoters include promoters from genes such as cytochrome P450 genes, heat shock protein genes, metallothionein genes, hormone-inducible genes, such as the estrogen gene promoter, and the like. The promoter can also be tissue specific. Examples of such promoters that may be used with the expression vectors of the invention include promoters from the liver fatty acid binding (FAB) protein gene, specific for colon epithelial cells; the insulin gene, specific for pancreatic cells; the transphyretin, α1-antitrypsin, plasminogen activator inhibitor type 1 (PAI-1), apolipoprotein AI and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells. The selected promoter may also be linked to other sequences rendering it inducible or tissue-specific. For example, the addition of a tissue-specific enhancer element upstream of a selected promoter may render the promoter more active in a given tissue or cell type.

In addition to promoter/enhancer elements, expression vectors of the invention may further comprise a suitable terminator. Such terminators include, for example, the human growth hormone terminator, or, for yeast or fungal hosts, the TPII (Alber & Kawasaki, J. Mol. Appl. Genet. 1:419-34, 1982) or ADH3 terminator (McKnight et al., EMBO J. 4: 2093-2099, 1985). Vectors useful for the invention may also comprise polyadenylation sequences (e.g., the SV40 or Ad5E1b poly(A) sequence), and translational enhancer sequences (e.g., those from Adenovirus VA RNAs). Further, a vector useful for the invention may encode a signal sequence directing the variant venom polypeptide to a particular cellular compartment or, alternatively, may encode a signal directing secretion of the polypeptide.

Depending on the specific vector used for expressing the fusion polypeptide, various known cells or cell lines can be employed in the practice of the invention. The host cell can be any cell into which recombinant vectors encoding a variant venom peptide or polypeptide of the invention may be introduced and wherein the vectors are permitted to drive the expression of the polypeptide. It may be prokaryotic, such as any of a number of bacterial strains, or may be eukaryotic, such as yeast or other fungal cells, insect or amphibian cells, or mammalian cells including, for example, rodent, simian or human cells. Cells expressing the fusion polypeptides of the invention may be primary cultured cells, for example, primary human fibroblasts or keratinocytes, or may be an established cell line, such as NIH3T3, HEK293, HEK293T HeLa, MDCK, WI38, or CHO cells. Further, mammalian cells useful for expression of the fusion polypeptides of the invention may be phenotypically normal or oncogenically transformed. These include, e.g., tumor cells such as non-small cell lung cancer (NSCLC) cell line A549 or Eμ-Myc lymphoma cells as exemplified herein. Any other primary mammalian cells or transformed tumor cells may also be used in the practice of the reporter fusion polypeptides of the invention. The skilled artisans can readily establish and maintain a chosen host cell type in culture that expresses the fusion polypeptide. Many other specific examples of suitable cell lines that can be used in expressing the fusion polypeptides are described in the art. See, e.g., Smith et al., J. Virol. 46:584, 1983; Engelhard, et al., Proc. Natl. Acad. Sci. USA 91:3224, 1994; Logan and Shenk, Proc. Natl. Acad. Sci. USA 81:3655, 1984; Scharf, et al., Results Probl. Cell Differ. 20:125, 1994; Bittner et al, Methods Enzymol. 153:516, 1987; Van Heeke & Schuster, J. Biol. Chem. 264:5503, 1989; Grant et al., Methods Enzymol. 153:516, 1987; Brisson et al., Nature 310:511, 1984; Takamatsu et al., EMBO J. 6:307, 1987; Coruzzi et al., EMBO J. 3:1671, 1984; Broglie et al., Science 224:838, 1984; Winter and Sinibaldi, Results Probl. Cell Differ. 17:85, 1991; and Weissbach and Weissbach (1988) Methods for Plant Molecular Biology, Academic Press, New York, pp 421-463.

The venom derived polypeptide-expressing vectors may be introduced to selected host cells by any of a number of suitable methods known to those skilled in the art. For example, vectors expressing the polypeptide may be introduced into appropriate bacterial cells by infection, in the case of E. coli bacteriophage vector particles such as lambda or M13, or by any of a number of transformation methods for compatible plasmid vectors or for bacteriophage DNA. For example, standard calcium-chloride-mediated bacterial transformation is still commonly used to introduce naked DNA to bacteria (Sambrook et al., Molecular Cloning: A Laboratory Manual, Cold Spring Harbor Press, N.Y., (3^{rd} ed., 2000)), but electroporation may also be used (Brent et al., supra).

For the introduction of constructs expressing the venom derived polypeptide into yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisia, for example, the cells are treated with lithium acetate to achieve transformation efficiencies of approximately 104 colony-forming units (transformed cells)/μg of DNA. Transformed cells are then isolated on selective media appropriate to the selectable marker used. Alternatively or additionally, plates or filters lifted from plates may be scanned for fluorescence and luciferase-mediated bioluminescence to identify transformed clones with the fusion polypeptide-encoding constructs.

For the introduction of venom derived polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al., supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

### V. Therapeutic and Diagnostic Applications

The Kv1.3 channel is expressed on all subsets of T cells and B cells, but effector memory T cells and class-switched memory B cells are particularly dependent on Kv1.3 (Wulff et al., J. Immunol. 173:776, 2004). Kv1.3 is overexpressed in Gad5/insulin-specific T cells from patients with new onset type 1 diabetes, in myelin-specific T cells from MS patients, in T cells from the synovium of rheumatoid arthritis patients (Beeton et al., Proc. Natl. Acad. Sci. USA 103:17414-9, 2006), in breast cancer specimens (Abdul et al., Anticancer Res 23:3347, 2003), and in prostate cancer cell lines (Fraser at al., Pflugers Arch. 446:559, 2003). Blockade of Kv1.3 channels in effector-memory T cells suppresses calcium signaling, cytokine production (interferon-gamma, interleukin 2) and cell proliferation. In vivo, Kv1.3 blockers paralyze effector-memory T cells at the sites of inflammation and prevent their reactivation in inflamed tissues. Positive outcomes in animal models with Kv1.3 blockers have also been described in hypersensitivity models to ovalbumin and tetanus toxoid, models for multiple sclerosis such as rat adoptive-transfer experimental autoimmune encephalomyelitis (AT-EAE) model, inflammatory bone resorption model, as well as animal models for arthritis, obesity, diabetes and metabolic diseases.

The invention accordingly provides various therapeutic applications of the Kv1.3 blocking variant venom peptides or polypeptides described herein. They can be used in vitro or in vivo for blocking or suppressing undesired Kv1.3 activity in a biological tissue or a target cell (e.g., a lymphocyte) with overexpressed Kv1.3. They can also be used in treating, or ameliorating symptoms associated with, any conditions or disorders that are associated with or mediated by undesired or aberrant Kv1.3 ion channel activity or overexpression. For example, in patients with multiple sclerosis (MS), disease-associated myelin-specific T cells from the blood are predominantly co-stimulation-independent effctor-memory T cells that express high levels of Kv1.3 channels. T cells in MS lesions in postmortem brain lesions are also predominantly effector-memory T cells that express high levels of the Kv1.3 channel. Thus, the Kv1.3 blocking polypeptides of the invention can be used for selectively suppressing Kv1.3 channel activity in these cells and for treating diseases associated with undesired T cell activation. For example, selective blockade of Kv1.3 channel in effector-memory T cells can be effective for autoimmune diseases without compromising the protective immune response. The Kv1.3 blocking peptides or polypeptides of the invention can also be used for treating various other conditions or disorders that are mediated with undesired Kv1.3 ion channel activity or overexpression. For example, other than treating inflammatory and autoimmune diseases, the therapeutic methods of the invention can also be used for treating diabetes, obesity or cancers.

Typically, the therapeutic methods of the invention involve contacting a target cell or tissue expressing Kv1.3 with a therapeutically effective amount of a Kv1.3 blocking peptide or polypeptide of the invention, or a pharmaceutically acceptable salt thereof. For in vivo treatment of a disease or disorder that is associated with or mediated by undesired Kv1.3 channel activity or overexpression, the subject in need of treatment is typically administered with a pharmaceutical composition containing an effective amount of the Kv1.3 blocking peptide or polypeptide. In some embodiments, a purified Kv1.3 blocking agent of the invention is contacted with the target cell or administered to the subject in need of treatment. In some other embodiments, a polynucleotide or vector that expresses the Kv1.3 blocking polypeptide is introduced into the target cell or administered to the subject. In the latter embodiments, expression of the Kv1.3 blocking polypeptide in the target cell or inside the body of the subject can be either constitutive or controllable (e.g., via various expression control mechanisms such as an inducible promoter).

Many diseases or disorders associated with or mediated by undesired Kv1.3 channel activity/overexpression are suitable for treatment with the therapeutic methods of the invention. In some embodiments, the therapeutic methods of the invention are directed to treating or preventing diseases that are associated with aberrant or undesired T cell activation. The methods are intended to inhibit and reduce T cell activation by at least 20, 30%, 40%, 50%, 60%, 70%, 80%, 90%, or 100%. T cell activation can be measured by well-known methods, such as measuring reduction of IL-2 production by T cells. The therapeutic methods of the invention can also be used for the prophylaxis of diseases or conditions that are mediated by or associated with undesired Kv1.3 channel activity or overexpression. The Kv1.3 blocking peptide or polypeptide of the invention can also be used in the preparation of a medicament for such treatment, wherein the medicament is prepared for administration in dosages defined herein.

Examples of specific diseases or disorders suitable for the therapeutic methods of the invention include, e.g., inflammatory conditions, allergies and allergic conditions, hypersensitivity reactions, autoimmune diseases, severe infections, and organ or tissue transplant rejection. Specific examples of such diseases or disorders include rheumatoid arthritis (RA), ankylosing spondylitis, psoriatic arthritis, osteoarthritis, osteoporosis, uveitis, inflammatory fibrosis, scleroderma, lung fibrosis, cirrhosis, inflammatory bowel disease, Crohn's disease, ulcerative colitis, asthma, allergic asthma, allergies, Chronic Obstructive Pulmonary Diseases (COPD), multiple sclerosis, psoriasis, contact-mediated dermatitis, systemic lupus erythematosus (SLE) and other forms of lupus, diabetes, type I diabetes, obesity, cancer, lupus, restenosis, systemic sclerosis, scleroderma, glomerulonephritis, Sjogren syndrome, inflammatory bone resorption, transplant rejection, and graft-versus-host disease.

Therapeutic efficacy of the Kv1.3 blocking peptides or polypeptides of the invention for a particular disease can be examined with methods that are based on or adapted from any suitable in vitro or animal models that are well known in the art. These include models such as collagen-induced arthritis (CIA) model, diet-induced obesity model, the 2,4,6-trinitrobenesulfonic acid/ethanol (TNBS)-induced colitis model or the oxazalone model, which induce chronic inflammation and ulceration in the colon (Neurath et al., Intern. Rev. Immunol. 19:51-62, 2000), the adoptive transfer model of naive CD45RB^{high} CD4 T cells to RAG or SCID mice, the donor naive T cells attack the recipient gut causing chronic bowel inflammation and symptoms similar to human inflammatory bowel diseases (Read and Powrie, Current Protocols in Immunology, Chapter 15 unit 15.13, 2001), ovalbumin challenge model and methacholine sensitization models (Hessel et al., Eur. J. Pharmacol. 293:401-12, 1995). Other well-known animal models that can be used in the practice of the methods of the invention include hypersensitivity models to ovalbumin and tetanus toxoid (Beeton et al., Mol. Pharmacol. 67:1369, 2005; Koo et al., Clin. Immunol. 197:99, 1999), models for multiple sclerosis such as rat adoptive-transfer experimental autoimmune encephalomyelitis (AT-EAE) model (Beeton et al, Proc. Natl. Acad. Sci. USA 103:17414-9, 2006), inflammatory bone resorption model (Valverde et al., J. Bone. Mineral. Res. 19:155, 2004), models for arthritis (Beeton et al., Proc. Natl. Acad. Sci. USA 103: 17414, 2006; Tarcha et al., J. Pharmacol. Exper. Therap. 342: 642, 2012) and obesity, diabetes and metabolic diseases (Xu et al., Hum. Mol. Genet. 12:551, 2003; Xu et al., Proc. Natl. Acad. Sci. USA 101: 3112, 2004).

The invention additionally provides diagnostic methods for using the Kv1.3-binding peptides or polypeptides described herein (e.g., SEQ ID NO:4 or SEQ ID NO:1) in detecting and quantifying Kv1.3 expression in various target cells or biological tissues of interest. In related embodiments, the invention provides methods for diagnosing in a subject a disease or disorder that is mediated by or associated with abnormal Kv1.3 expression. In some of these embodiments, the diagnostic methods of the invention are directed to detecting a high or overexpressed Kv1.3 level in a target cell (e.g., lymphocytes) in subjects afflicted with or at risk of developing a disease that is associated with or mediated by overexpressed Kv1.3 (e.g., an autoimmune disease). A high or overexpressed Kv1.3 level refers to the number of Kv1.3 channel on each target cell that is substantively higher (e.g., at least 25%, 50%, 75%, or 95% more) than the normal or mean number of Kv1.3 channels typically observed in the same type of cells from control healthy subjects of the same or similar attributes. Thus, as exemplification, a high or overexpressed Kv1.3 level in human T lymphocytes refers the presence of at least about 400, 500 or 600 channels/cell.

As noted above, upregulated Kv1.3 expression is implicated in the development of a number of diseases. For example, T lymphocytes with unusually high expression level of Kv1.3 have been associated with the pathogenesis of autoimmune disorders such as multiple sclerosis. Overexpressed Kv1.3 expression in microglia is also associated with Alzheimer's disease in human patients. The diagnostic methods of the invention can be used in subjects to diagnose or confirm any of these diseases that are associated with overexpressed Kv1.3 levels. The variant venom peptides or polypeptides described herein are capable of binding to Kv1.3 with strong specificity and affinity. In addition, unlike Kv1.3-specific antibodies, the Kv1.3 binding agents of the invention can bind to the outer vestibule of Kv1.3 and can therefore reach their binding pocket in live intact lymphocytes. Thus, when labeled with or conjugated to an appropriate detection agent (e.g., fluorophores), the Kv1.3 binding agents of the invention provide effective molecular probes to detect and quantify Kv1.3 expression in cells of interest, e.g., lymphocytes. A detected Kv1.3 expression level in a cell or tissue sample (e.g., lymphocytes) obtained from a candidate subject or suspected patient can be compared with a normal or control Kv1.3 expression level in the same cell or tissue from healthy subjects. This provides an indication whether the candidate subject has or is at risk of developing a disease or disorder that is associated with or mediated by high Kv1.3 expression (e.g., an autoimmune disease).

Labeling a Kv1.3 binding polypeptide of the invention with a suitable labeling agent (e.g., a fluorophore) for use in the diagnostic methods of the invention can be readily carried out in accordance with techniques well established in the art. For example, there have been many studies on fluorophore-labeled ion channel-binding peptides and their use in probing ion channel expression in cells via flow cytometry. See, e.g., Joe et al., J. Neurosci. 13:2993-3005, 1993; Jones et al., Science 244:1189-1193, 1989; Benke et al., Proc. Natl. Acad. Sci. U.S.A 90:7819-7823, 1993; Pragl et al., Bioconjugate Chem. 13:416-425, 2002; Freudenthaler et al., Histochem. Cell Biol. 117:197-202, 2002; Beeton et al., J. Biol. Chem. 278:9928-37, 2003; and Pennington et al., Mol. Pharmacol. 75:762-773, 2009. Other than fluorescence based probe labeling and Kv1.3 detection, the diagnostic methods of the invention can also employ other means for labeling the Kv1.3-binding agents described herein and corresponding assays for detecting and quantifying Kv1.3 expression. For example, the peptide probe can be labeled with a radioactive material as described in the art, e.g., Tarcha et al., J. Pharmacol. Exp. Ther. 342: 642-653, 2012. Upon contacting a biological sample (e.g., a T cell sample) with the labeled peptide probe, Kv1.3 expression level in the sample can be readily quantified based on the amount of radioactivity in the sample. Any of the methods described in the art can be modified for use in the practice of the diagnostic methods of the invention.

### VI. Pharmaceutical Compositions and Kits

The Kv1.3 blocking variant venom peptides or polypeptides of the invention can be directly administered under sterile conditions to the subject to be treated. The polypeptides can be administered alone or as the active ingredient of a pharmaceutical composition. Therapeutic composition of the present invention can be combined with or used in association with other therapeutic agents. For example, a subject may be treated with a variant venom peptide of the invention along with another known Kv1.3 blocking agent. Examples of the latter agent include small molecule compounds, e.g., PAP-1, correolide, benzamides, CP339818, progesterone, and the anti-lepromatous drug clofazimine. They also include other venom related peptides from scorpions (ADWX1, OSK1, margatoxin, kaliotoxin, charybdotoxin, noxiustoxin, anuroctoxin) and sea anemone (ShK, ShK-F6CA, ShK-186, ShK-192, BgK). In addition to pharmaceutical compositions, the invention also provides kits for carrying out the diagnostic methods described herein. The kits typically contain one or more Kv1.3 blocking agents described herein, optionally also some related reagents for using the Kv1.3 blocking agents for detecting and quantifying Kv1.3 expression in target cells or tissues (e.g., fluorescence labels), as well as instructions on how to use the Kv1.3 blocking agents and other components in the kits.

Pharmaceutical compositions of the present invention typically comprise at least one active ingredient (e.g., a Kv1.3 blocking polypeptide disclosed herein) together with one or more acceptable carriers thereof. Pharmaceutically carriers enhance or stabilize the composition, or to facilitate preparation of the composition. Pharmaceutically acceptable carriers are determined in part by the particular composition being administered, as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the Kv1.3 blocking peptides or polypeptides can be complexed with carrier proteins such as ovalbumin or serum albumin prior to their administration in order to enhance stability or pharmacological properties.

The pharmaceutical compositions of the invention can be administered to subjects in need of treatment in a variety of ways. These include both local and systemic administrations. Specific examples of routes of administration include, without limitation, oral, parenteral (including subcutaneous, intravenous, intramuscular and intraperitoneal), rectal, vaginal, dermal, transdermal (topical), transmucosal, intrathoracic, intrapulmonary and intranasal (respiratory) routes. The means of administration may be by injection, using a pump or any other appropriate mechanism.

The pharmaceutical compositions can be prepared in various forms, such as granules, tablets, pills, suppositories, capsules, suspensions, salves, lotions and the like. The concentration of therapeutically active compound in the formulation may vary from about 0.1 to 100% by weight. Therapeutic formulations are prepared by any methods well known in the art of pharmacy. The therapeutic formulations can be delivered by any effective means which could be used for treatment. See, e.g., Goodman & Gilman's The Pharmacological Bases of Therapeutics, Hardman et al., eds., McGraw-Hill Professional (10^{th} ed., 2001); Remington: The Science and Practice of Pharmacy, Gennaro, ed., Lippincott Williams & Wilkins (20^{th} ed, 2003); and Pharmaceutical Dosage Forms and Drug Delivery Systems, Ansel et al. (eds.), Lippincott Williams & Wilkins (7^{th} ed., 1999).

The therapeutic formulations can conveniently be presented in unit dosage form and administered in a suitable therapeutic dose. A suitable therapeutic dose can be determined by any of the well-known methods such as clinical studies on mammalian species to determine maximum tolerable dose and on normal human subjects to determine safe dosage. Except under certain circumstances when higher dosages may be required, the preferred dosage of a Kv1.3 blocking polypeptide usually lies within the range of from about 0.001 to about 1000 mg, more usually from about 0.01 to about 500 mg per day. The dosage to be administered to a subject can be any amount appropriate to eliminate or ameliorate at least one symptom associated with the disease or disorder to be treated (e.g., diabetis or obesity). Some factors that determine appropriate dosages are well known to those of ordinary skill in the art and may be addressed with routine experimentation. For example, determination of the physicochemical, toxicological and pharmacokinetic properties may be made using standard chemical and biological assays and through the use of mathematical modeling techniques known in the chemical, pharmacological and toxicological arts. The therapeutic utility and dosing regimen may be extrapolated from the results of such techniques and through the use of appropriate pharmacokinetic and/or pharmacodynamic models. Other factors will depend on individual patient parameters including age, physical condition, size, weight, the condition being treated, the severity of the condition, and any concurrent treatment. The dosage will also depend on the polypeptide chosen (e.g., SEQ ID NO:1 or SEQ ID NO:4) and whether prevention or treatment is to be achieved, and if the polypeptide is chemically modified. Such factors can be readily determined by the animal models described herein or other animal models or test systems that are available in the art.

The Kv1.3 blocking polypeptides or peptides provided herein can be administered in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents can be essentially continuous over a pre-selected period of time or may be in a series of spaced doses. For example, therapeutic composition containing a Kv1.3 blocking polypeptide of the invention can be administered to the subject once or multiple times (e.g., twice or three times) a day, once or multiple times every week or every two weeks, or once or several times every month. Depending on the specific subjects and the condition to be treated, the administration can last for varying length of time, e.g., a few days, a few weeks, a few months, a few years or longer. In general, the amount of a given Kv1.3 blocking included in a unit dose can vary widely. Thus, the amount of the drug to be administered in a unit dosage can be, e.g., at least about 0.01 mg/kg to about 500, 750 or 1000 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. Measured alternatively, the unit dosage to be administered can be in the range of at least about 0.5 nmol/kg to about 25 or 50 mmol/kg, at least about 1 nmol/kg to about 5 or 10 mmol/kg, or at least about 2.5 nmol/kg to about 500 or 1000 nmol/kg. In addition, when the drug is administrated more frequently (e.g., daily administration), a lower unit dosage may be employed. Conversely, in some chronic treatment that involve less frequent administration of the drug (e.g., weekly or biweekly administration), a higher unit dosage can be used. Further, other than a fixed dosage throughout the treatment regimen or treatment process, the subject may also be administered with varying dosages along the duration of any treatment regimen or the entire treatment process. For example, a treatment regimen can employ daily or weekly dosages that are increasingly higher as the treatment regimen progresses. Alternatively, a treatment regimen can start with a higher daily or weekly dosage at the beginning, and gradually decrease the dosage administered in the following days or weeks. In some other embodiments, a treatment process can contain multiple treatment regimens that employ differing dosages.

The preferred dosage and mode of administration of a Kv1.3 blocking polypeptide of the invention can vary for different subjects, depending upon factors that can be individually reviewed by the treating physician, such as the condition or conditions to be treated, the choice of composition to be administered, including the particular Kv1.3 blocking, the age, weight, and response of the individual subject, the severity of the subject's symptoms, and the chosen route of administration. As a general rule, the quantity of a Kv1.3 blocking agent administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are provided to further illustrate the invention but not to limit its scope. Other variants of the invention will be readily apparent to one of ordinary skill in the art and are encompassed by the appended claims.

### Example 1 Construction of Selection System for Identifying Ion Channel Blocker

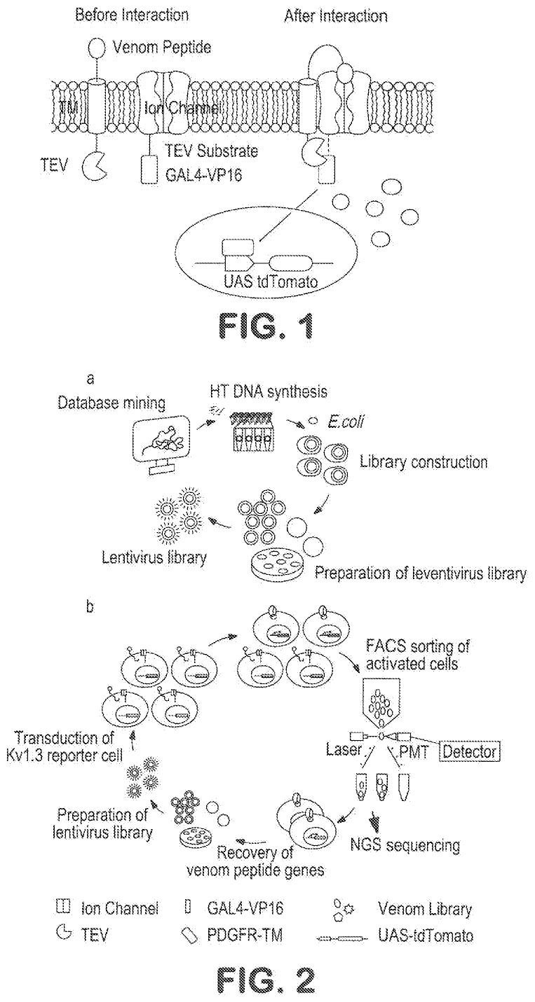

For the initial proof of concept, interactions between the Kv1.3 channel and the characterized venom peptides were studied using an autocrine format and proximity based assay. To construct the system, the venom peptides were constructed where the platelet derived growth factor receptor transmembrane domain (PDGFR-TM) anchored the venom(s) of interest to the plasma membrane. The Tobacco Etch Virus (TEV) protease was added to the cytoplasmic face of the PDGFR-TM domain. The reason for using the TEV protease is its high sequence specificity. The cytoplasmic face of the voltage gated potassium channel Kv1.3 was coupled to an optimized TEV substrate sequence and an artificial transcription factor GAL4-VP16. One now had venoms bearing a protease and target channels bearing a substrate for the protease. When the Kv1.3 channel and venom peptides interacted they brought the enzyme and its substrate into proximity and catalytic cleavage of the substrate sequence occurred. The released transcription factor entered the nucleus and activated the expression of the fluorescent protein reporter gene (FIG. 1). The nature of the vectors is described in FIG. 6. The method is general and only requires proximity of venom peptides and the channel such that the number of productive collisions is increased (Zhang et al., Biochem. Biophys. Res. Commun. 454, 251-255, 2014).

We generated a Kv1.3 reporter cell line in which the Kv1.3 channel fusion protein was expressed at low levels and the reporter vector containing the upstream activation sequence (UAS) for transcription activation of the expression of the reporter gene tdTomato was integrated into the genome. The Kv1.3-blocking peptide ShK and Anuroctoxin were cloned into the pLigand vector and corresponding lentivirus were prepared. Lentivirus encoding either peptide stimulated the Kv1.3 reporter cells to express tdTomato fluorescent protein while the negative control lentivirus encoding GLP-1 receptor targeting Exendin-4 or GABA_{B} receptor targeting Vc1.1 exhibited no activity (FIG. 7).

### Example 2 Validation of Selection System by Selecting Natural Venom Peptides

After the initial experiments showing that the selection system was functional, we next selected Kv1.3 targeting peptides from a natural venom peptide library. Cysteine knot peptides were identified from the ATDB (He et al., Nucleic acids research 2008, 36, D293-297) and ArachnoServer (Herzig et al., Nucleic acids research 2011, 39, D653-657) based on 3 criteria (FIG. 2a). First, the toxin should be less than 42 amino acids long. Secondly, the toxin peptides should contain at least 6 cysteines indicating the peptide featured a cysteine knot structure. Lastly, redundant peptides were removed. As a result, 589 natural venom toxins and their variants were extracted after data mining of the databases.

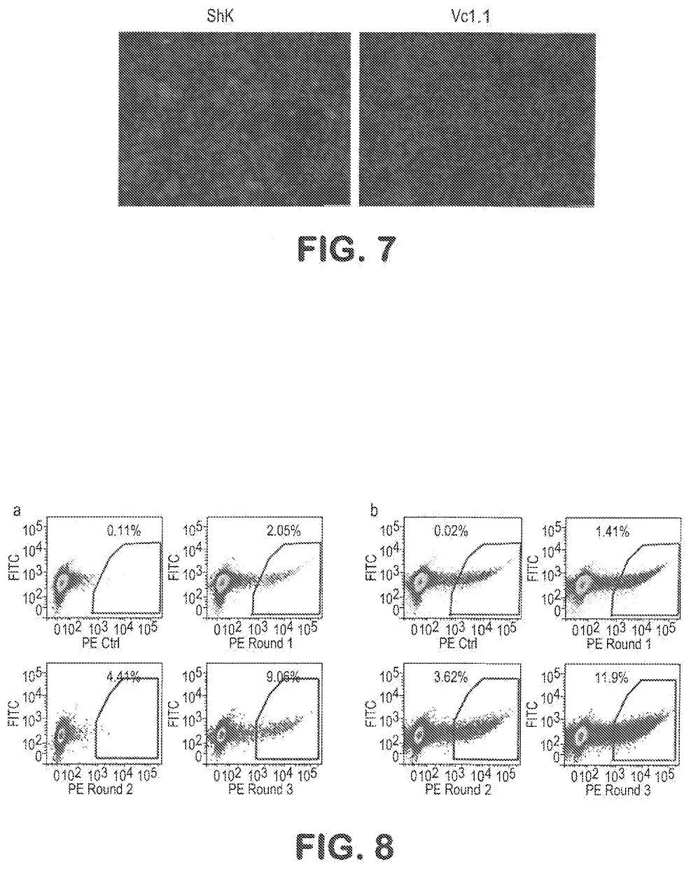

The key to our method is that it utilizes autocrine based selection, and each cell becomes a reporter system unto itself. Kv1.3 reporter cells were transduced with a lentiviral library of natural venom peptides at a multiplicity of infection (MOI) equal to 1 so that on the average every cell in the culture received one virion. After infection, a venom peptide fusion protein and Kv1.3 fusion protein were coexpressed in the plasma membrane of the same cell. Only when the venom peptide interacted with Kv1.3, were the TEV protease and its substrate sequence brought into proximity, resulting in release of the transcription factor and expression of tdTomato. Two days post-infection, tdTomato-positive cells were sorted by FACS. The toxin genes were recovered from the sorted cells by PCR and cloned into pLigand vector to prepare an enriched lentiviral library for the next round selection (FIG. 2b). Significant enrichment was observed during three rounds of selection of the natural venom peptide library (FIG. 8a). To go beyond flow cytometry data and to understand the selection process in more detail, NGS sequencing was done and enrichment factor of the individual toxin in each round was calculated.

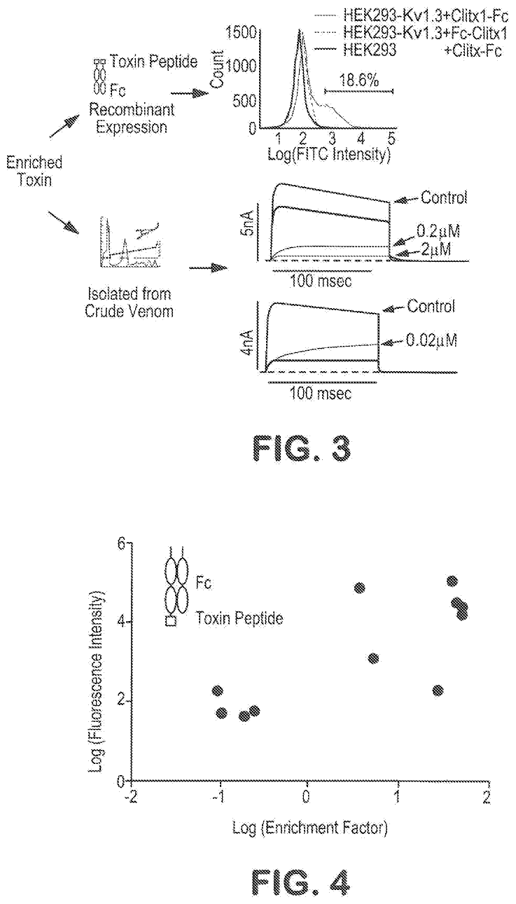

The NGS results are summarized in Table 1. Twenty five out of the twenty seven most enriched sequences are known blockers of the Kv1.3 channel (Table 1). There remained two peptides whose specificity was unknown. We tested the CllTx1 peptide for its ability to block the Kv1.3 channel. Because of the difficulties encountered when attempting chemical synthesis of cysteine knot peptides, we fused the CllTx1 peptide to the N-terminus of human IgG1 Fc fragment and generated the fusion protein by recombinant expression. Immunofluorescence staining of Kv1.3 expressing HEK293F cells showed that the CllTx1-Fc fusion protein bound to the Kv1.3 channel (FIG. 3). The peptide was also purified from crude venom of scorpion Centruroides limpidus limpidus by HPLC as described in Martin et al., Biochem J 1994, 304 (Pt 1), 51-56. This authentic venom peptide strongly blocked the Kv1.3 channel (FIG. 3). The other toxin Aek was previously classified as a potassium channel toxin based on its sequence similarity with AsKS, BgK and ShK. Thus, the selection method was not only validated by reproducing of toxins known to bind to the input target but was also able to identify new toxins whose target specificity was previously unknown.

### Example 3 Selecting Venom Peptide Variants that Target Kv1.3 Channel

An equally important aspect of this method is that it allows one to select from venom peptide based combinatorial libraries to find variants with improved pharmacological properties. We demonstrated the utility of the method for protein engineering by generating potent and selective Kv1.3 blockers with long in vivo half-life.

ShK is the most widely-studied Kv1.3 targeting venom peptide and its synthetic analog ShK-186 has completed phase-1 trials for active plaque psoriasis. However, this molecule had a short in vivo half-live. Fusion to an IgG1 Fc is an established strategy to extend the half-life of therapeutic proteins. Thus, we engineered the venom peptide ShK as a fusion protein to the C-terminus of the human IgG1 Fc fragment. Although N-terminus of the venom faces away from the channel pore, the potency of a toxin peptide may be decreased when it is fused to C terminus of Fc fragment because of steric hindrance. To overcome this problem, the first 7 positions of ShK were randomized in order to re-orientate the Fc portion of the construct in a variety of ways. The diversity of this library was about one million members. The Kv1.3 reporter cell line was infected with the combinatorial library of Fc-ShK variants, and the variants that retained Kv1.3 binding ability were enriched by 3 rounds of selection (FIG. 8b). Variants from each round of selection were analyzed by NGS and the frequencies of variants were calculated.

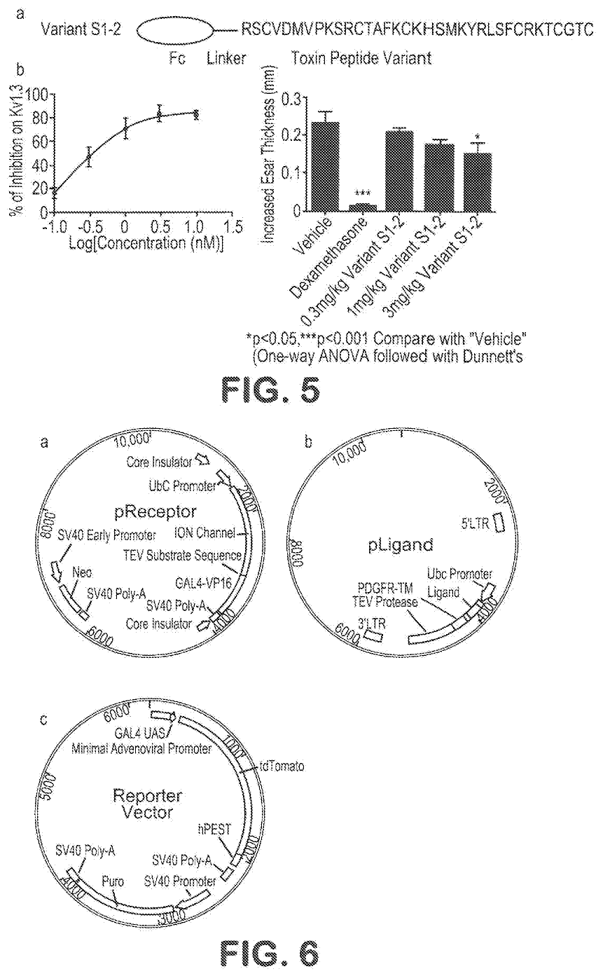

The NGS results are summarized in Table 2. The variegated region of each variant was extracted based on the known flanking sequences. The ratio between number of unique toxins and count of all reads represented the diversity of a selected library. Highly enriched variants versus those that were not enriched were chosen and their affinity to Kv1.3 were assessed and ranked. In general, the enrichment factor of a variant is positively correlated with its affinity to Kv1.3 (FIG. 4). Patch clamp studies showed that the highly enriched variant S1-2 (FIG. 5a) retains its potency as a blocker of the Kv1.3 channel (IC₅₀=158 pM) (FIG. 5b). To test the critical property of specificity, patch clamp analysis of S1-2 was also carried out on HEK293 cells expressing other Kv1 family members including Kv1.1 through Kv1.7 and the hERG channel. The wild type ShK exhibited comparable inhibition of Kv1.1, Kv1.6 and Kv1.3, whereas ShK variant S1-2 had no effect on any of the other channels (Table 3).

### Example 4 In Vivo Activity of Selected Venom Peptide Variant S1-2

Because the Kv1.3 channel is known to be involved in effector memory T cell activation, blocking it presents a novel therapeutic opportunity for autoimmune diseases such as psoriasis and rheumatoid arthritis. We studied the efficacy of the selected channel blocker in the DNFB induced rat delayed-type hypersensitivity model. Lewis rats were dosed with Fc-ShK variant S1-2 (0.3.1 and 3.0 mg/kg) subcutaneously and change of ear thickness was measured post DNFB challenge. Variant S1-2 showed a dose dependent reduction of inflammation. At the maximum dose of 3 mg/kg, variant S1-2 achieved 40% decrease in car thickness after DNFB challenge (FIG. 5c).

Many of the problems plaguing venom research can be circumvented by the method described in this report. Here, as with antibodies, venoms are collected in a combinatorial matrix that allows for expanded diversity and utilizes selection to identify functional molecules. Since the method relies only on bioinformatics and genetics for generation of the repertoire, one does not require the presence of captured venomous organisms and the synthetic problems are avoided. In addition, selection of a library of venom peptides by flow cytometry can significantly reduce the number of molecules for the expensive and time-consuming electrophysiology measurements. Another important feature of autocrine based selection system is that it allows one to study the target in its natural milieu of the plasma membrane.

TABLE 1
Statistics of NGS data of selection of the natural venom peptide library

| | | Mapped | Mapped |
|---|---|---|---|
| | | Natural | Natural |
| Selection | Count of | Venom | Venom |
| Round | Reads | Peptide Reads | Peptide Ratio |
| Original Library | 812,216 | 410,519 | 50.54% |
| Round 1 | 589,394 | 308,192 | 52.29% |
| Round 2 | 774,915 | 473,224 | 61.07% |
| Round 3 | 1,106,188 | 740,720 | 66.96% |

TABLE 2
Statistics of NGS data of selection of the Fc-ShK combinatorial library

| | | Count of | Percentage | Number of | |
|---|---|---|---|---|---|
| | | ShK | of ShK | Unique | |
| Selection | Count of | Variants | Variants | Variants | Diversity |
| Round | Reads (a) | (b) | (b/a) | (c) | (c/b) |
| Library | 337,627 | 269,525 | 79.83% | 135,506 | 0.50 |
| Round 1 | 230,158 | 200,461 | 87.10% | 53,778 | 0.27 |
| Round 2 | 283,815 | 258,061 | 90.93% | 32,722 | 0.13 |
| Round 3 | 398,136 | 366,880 | 92.15% | 33,418 | 0.09 |

TABLE 3
Selectivity of the variant S1-2 on members of
Kv Family and hERG channel.

| Channels | Current block at 30 nM S1-2 (%) |
|---|---|
| Kv1.1 | 0.09 ± 0.16 |
| Kv1.2 | 2.33 ± 4.03 |
| Kv1.3 | 91.88 ± 8.39* |
| Kv1.4 | 0.79 ± 1.12 |
| Kv1.5 | 4.51 ± 3.58 |
| Kv1.6 | 4.05 ± 3.95 |
| Kv1.7 | 5.19 ± 3.80 |
| hERG | 2.44 ± 2.78 |

*10 nM variant S1-2 was tested on Kv1.3 while the concentration of 30 nM was used for other channels.

### Example 5 Materials and Methods

This Example describes some materials and methods that were employed in exemplifying the make and use of the polypeptides of the invention.

Cell lines: The HEK293 (ATCC cat No. CRL-1573) cell line was maintained in DMEM containing 10% (vol/vol) FBS, penicillin and streptomycin and transfected using Lipofectamine 2000. The HEK293F (Life technologies R790-07) cell line was maintained in Freestyle 293 Expression Media with 4 mM GlutaMAX and transfected using FreeStyle MAX Reagent.

Generation of the Kv1.3 reporter cell line for the proximity based assay: HEK293 cells were transfected with the reporter vector (FIG. 6) and selected with 3 μg/mL puromycin for two weeks to generate UAS-tdTomato HEK293 cells. The UAS-tdTomato HEK293 cells were transfected with pReceptor vector to express Kv0.3-TEV substrate sequence-Transcription factor GAL4-VP16 fusion protein. The cells were selected with 800 μg/mL geneticin for two weeks.

Construction of the natural venom peptide lentiviral library: The natural venom toxin genes were extracted from Animal Toxin Database (ATDB Version 2.5, updated 2010 Oct. 5) (He et al., Nucleic acids research 2008, 36, D293-297) and ArachnoServer 2.0 (Herzig et al., Nucleic acids research 2011, 39, D653-657). Toxin peptides were identified based on the following criteria. First, the toxin should be less than 42 amino acids long. Secondly, the toxin peptides should contain at least 6 cysteines. At last, the redundant peptides were removed. As a result, there were 589 natural venom toxins after data mining. All peptide sequences were back translated to DNA sequences. Varied numbers of GGC GGA GGT GGA AGC (SEQ ID NO:8), which encoded GGGGS (SEQ ID NO:9) linker, were added to the 3′ end of toxin DNA sequences to extend DNA length to 108-126 bases. At last, 5′ end flank sequence (TCTTGCACTTGTCACGAATTCG) (SEQ ID NO: 10) and 3′ end flank sequence (GCTAGCGTCGACTACGGAGATG) (SEQ ID NO: 11) were added to the toxin DNA sequences. Oligonucleotides were synthesized on the CustomArray 12K array and amplified by nested PCR using primers based on flank sequences. The amplified genes were cloned into the pLigand vector by enzyme digestion cloning method (FIG. 6) where the venom peptide was fused to the N-terminus of the platelet-derived growth factor receptor transmembrane domain (PDGFR-TM; amino acids 514-561) while the TEV protease was fused to the C-terminus of PDGFR-TM.

Construction of the combinatorial venom peptide library: Two combinatorial libraries were constructed. The N-terminal 7 amino acids RSCIDTI of ShK (Q16K) (Murray et al., J. Med Chem. 2015, 58, 6784-6802) in Fc-ShK were replaced with X2CX4 for library 1 or X2CXDX3 for library 2 (X=any amino acid). The library construction strategy and all primer sequences for library construction are provided in FIG. 8.

The diversified ShK fragment was amplified by PCR using NNK degenerate primers (N=any nucleotide, K=G/T, NNK codes for any amino acid). The amplified products were digested with EcoRI/NheI and subcloned into pLigand vector. The ligation was transformed into XL1-Blue electroporation competent cells by electroporation. Plate 1 uL, 10 uL and 100 uL and count the colonies on each plate and calculate the total number of transformants which was equal to the size of the library.

Preparation of the Lentiviral library: HEK293FT cells were of 80-90% confluent at the time of transfection. Cells were transfected with a DNA mixture of equal amounts of lentivirus library vector, packaging plasmid pCMVD8.9 and envelop plasmid pVSVg. The medium was changed the next day and virus was collected 2 days post-transfection. The supernatants were centrifuged at 1000×g for 5 min to remove cell debris and filtered through 0.45 μM cellulose acetate filters. The titer of the lentivirus preparation was determined using Lenti-X p24 ELISA (Clontech). P24 values can be used to determine the relative virus titers. To calibrate a relationship between p24 levels and infectivity, p24 levels of GFP encoding lentivirus for different MOIs were determined.

Autocrine based selection: Kv1.3 reporter cells were plated so that the cells were 20% confluent on the next day. The cells were infected with the venom peptide lentiviral library in complete media containing 5 μg/ml Polybrene. The amount of virus used for MOI equal to 1 was based on the relationship between p24 levels and infectivity. To ensure reproducible and reliable results for selection of pooled libraries, it is critical that that sufficient Kv1.3 reporter cells (20-million) were infected to maintain sufficient representation of each member in the library. Two days post-infection cells were detached using Accutase (Innovative cell technologies) and single cell suspensions were prepared by passing the detached cells through the 70 μm strainer. The tdTomato-positive cells were sorted using a MoFlo Astrios fluorescence-activated cell sorter (Beckman Coulter). The genes encoding the peptide sequences were recovered directly from sorted cells by PCR and cloned into lentiviral vectors to construct enriched library for the next round selection. Three iterative rounds of selection were carried out.

NGS sequencing and bioinformatics analysis: Venom genes were amplified from plasmids after each round using a different barcode for each round. Sequencing was performed on an Illumina sequencing platform. An in-house bioinformatics protocol was developed to process and analyze the NGS data.

The Illumina sequencing platform generated reads of 150 bp raw data. A raw data filtering process generated clean data with high quality. In particular, paired reads were removed when reads contained more than 10% of N (N means base can't be determined) or the quality value of over 50% bases of the read were below 5. Then the clean data processing was performed in CLC Genomics Workbench 8.5.1. First, paired reads were assembled, resulting in sequences containing the whole venom toxin. Secondly, sequences were assigned to different selection rounds based on the barcodes. Third, DNA sequences were translated into peptide sequences. For the natural venom peptide library, sequencing data should exactly match the peptide sequences in the natural venom toxin library. For the combinatorial library, the variegated region was extracted based on the known flanking sequences. The count of individual natural venom peptide or variant was normalized to the total read number of each round. The enrichment factor was calculated for individual venom peptide or variant by dividing its normalized count in each selection round (round 1 to 3) by its normalized count in the original library (round 0).

Expression and purification of the toxin Fc fusions: The toxin genes were fused to the N-terminus or C-terminus of the Fc portion of human IgG1 by overlap PCR and cloned into the pFUSE protein expression vector (InvivoGen). The wt Fc toxin fusion expression vector was transfected into HEK293F cells to generate bivalent Fc toxin fusions.

Efficient pairing Fc-ShK variant with Fec to generate monovalent Fc/Fc-ShK variant was achieved by the knob-into-hole approach. The ShK variant was fused to the C-terminus of Fe with the ‘knob mutation’ T366Y and the paired Fc contained a ‘hole mutation’ Y407T. To generate monovalent Fc/Fc-ShK variants, the knobs-into-holes plasmids were co-transfected into HEK293F cells (Ridgway et al., Prot. Eng. 1996, 9, 617-621).

Fc fusion proteins were purified using HiTrap Protein A HP column using gradient elution (loading and wash buffer: PBS, pH 7.4, elution buffer: 0.1 M citric acid, pH 3.5) and polished using HiTrap Capto S using gradient elution (start buffer 50 mM NaAc, pH 5, elution buffer: 50 mM NaAc, 1 M NaCl, pH 5) with ÅKTA Avant chromatography system. The sample buffer was exchanged to PBS, pH 7.4 and stored at 4° C. The protein concentrations were measured by Quant-iT Protein Assay (Life technology) and >95% purity was determined by SDS-PAGE. Samples used in animal experiment contained 0.5-1.0 EU endotoxin per milligram of protein as determined by the Chromogenic LAL Endotoxin Assay (Genscript).

Electrophysiology recording: HEKA EPC 10 USB patch clamp amplifier (from HEKA Elektronik, Germany) was used for the whole cell recording. A cover slip with abundant single HEK293 Kv1.3 stable cells on the surface was removed and placed into a continuously perfused (approximately 1-2 ml/minute) recording chamber mounted on an inverted microscope. The Kv1.3 current was recorded from single cell using standard whole cell recording techniques. The cell was voltage clamped at a holding potential of −80 mV. Outward potassium currents were measured using the whole-cell configuration of the patch clamp technique at a test potential of 40 mV from a holding potential of −80 mV. Current records were acquired at 2-5 KHz and filtered at 1-2 KHz. 40-80% series resistance compensation was routinely applied. Currents were elicited once every 20 s and were allowed to stabilize for 5-10 min before recording. Samples were applied using an ALA 8 channel perfusion system (ALA Scientific Instruments Inc.). Test substance was applied to the same cell from low to high concentration.

Rat delayed-type hypersensitivity model: 40 male Lewis rats at 6-7 weeks old were randomly grouped to 5 groups (n=8). On day 0 (start of the in-life) and day 1, 100 μL 1% (wt/vol) DNFB in 4:1 acetone/olive oil was applied to the shaved dorsum for sensitization. On day 5, baseline right pinna thickness was measured and both sides of the right pinnae of all animals were challenged with 50 μL 0.5% (wt/vol) DNFB in 4:1 acetone: olive oil. 24 hours after the DNFB challenge, the ear thickness was measured. Fc-ShK variant S1-2 and PBS vehicle control were subcutaneously dosed 16 hours and 1 hour before DNFB challenge in a blinded fashion. 10 mg/kg dexamethasone P.O. once daily was used as a positive control.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. An isolated or recombinant polypeptide, comprising a variant venom peptide that consists of (1) at least the first 18 N-terminal residues of the sequence RSCVDMVPKSRCTAFKCKHSMKYRLSFCRKTCGTC (SEQ ID NO:4) or conservatively modified variants thereof, (2) the sequence RSCIDTIPKSRCTAFKCKHSMKYRLSFCRKTCGTC (SEQ ID NO:6) except for at least one amino acid substitution selected from the group consisting of I4V, T6M, and I7V, or (3) at least 31 contiguous residues that are at least 90% identical to SEQ ID NO:4.

2. The polypeptide of claim 1, which specifically binds to Kv1.3 ion channel.

3. The polypeptide of claim 1, wherein the variant venom peptide consists of at least the first 20, 22, 24, 26, 28, 30, 32 or 34 N-terminal residues of SEQ ID NO:4 or conservatively modified variants thereof.

4. The polypeptide of claim 1, wherein the variant venom peptide consists of SEQ ID NO:6 except for at least two amino acid substitutions selected from the group consisting of I4V, T6M, and I7V.

5. The polypeptide of claim 1, wherein the variant venom peptide consists of 35 contiguous residues that are at least 90% identical to SEQ ID NO:4.

6-7. (canceled)

8. The polypeptide of claim 1, wherein the variant venom peptide consists of an amino acid sequence as shown in SEQ ID NO:4.

9. The polypeptide of claim 1, further comprising an Fc domain of an IgG that is fused at the N-terminus or C-terminus of the variant venom peptide.

10. (canceled)

11. The polypeptide of claim 9, wherein the Fc domain is fused via a linker sequence.

12. (canceled)

13. The polypeptide of claim 11, wherein the Fc domain is fused at the N-terminus via the linker sequence.

14. The polypeptide of claim 13, comprising an amino acid sequence that is at least 90% identical to SEQ ID NO: 1.

15. (canceled)

16. An isolated or purified polynucleotide that encodes the polypeptide of claim 1.

17. An expression vector harboring the polynucleotide of claim 16.

18. A method for blocking or suppressing Kv1.3 ion channel activity in a cell, comprising contacting the cell with a therapeutically effective amount of the polypeptide of claim 1, thereby blocking or suppressing Kv1.3 ion channel activity in the cell.

19-22. (canceled)

23. A method of treating a subject afflicted with a disease or disorder that is associated with or mediated by undesired or aberrant Kv1.3 ion channel activity or overexpression, comprising administering to the subject a pharmaceutical composition comprising a therapeutically effective amount of the polypeptide of claim 1, thereby treating the disease or disorder in the subject.

24. The method of claim 23, wherein the disease or disorder is associated with undesired T cell activation.

25. The method of claim 23, wherein the subject is afflicted with an inflammatory disorder or an autoimmune disease.

26. A method for detecting overexpression of Kv1.3 ion channel in a target cell, comprising (a) contacting the cell with a polypeptide of claim 1, (b) detecting a signal indicative of a specific binding of the polypeptide to the target cell, and (c) comparing the detected signal to a baseline signal from binding of the polypeptide to a control cell; wherein a substantive departure of the detected signal from the baseline signal indicates overexpression of Kv1.3 in the target cell.

27. The method of claim 26, wherein the polypeptide is labeled with a fluorophore, and the specific binding of the polypeptide to the cell is detected by flow cytometry.

28. The method of claim 26, wherein the target cell is isolated from a biological sample from a subject suspected of having a disorder associated with overexpressed Kv1.3.

29. The method of claim 28, wherein the target cell is T lymphocyte, and the disorder is an autoimmune disease.


SEQUENCE LISTING (13 sequences)

SEQ ID NO: 1 - protein, 280 aa, Artificial sequence
  Synthetic sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGNSGGGGSGGGGSRSCVDMVPKSRCTAFKCKHSMKYRLSFCRKTCGTC

SEQ ID NO: 2 - protein, 232 aa, Artificial sequence
  Synthetic sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 13 aa, Artificial sequence
  Synthetic sequence
GNSGGGGSGGGGS

SEQ ID NO: 4 - protein, 35 aa, Artificial sequence
  Synthetic sequence
RSCVDMVPKSRCTAFKCKHSMKYRLSFCRKTCGTC

SEQ ID NO: 5 - skipped

SEQ ID NO: 6 - protein, 35 aa, Artificial sequence
  Synthetic sequence
RSCIDTIPKSRCTAFKCKHSMKYRLSFCRKTCGTC

SEQ ID NO: 7 - protein, 35 aa, Artificial sequence
  Synthetic sequence
LRCFDLLPKSRCTAFKCKHSMKYRLSFCRKTCGTC

SEQ ID NO: 8 - DNA, 15 bp, Artificial sequence
  Synthetic sequence
ggcggaggtggaagc

SEQ ID NO: 9 - protein, 5 aa, Artificial sequence
  Synthetic sequence
GGGGS

SEQ ID NO: 10 - DNA, 22 bp, Artificial sequence
  Synthetic sequence
tcttgcacttgtcacgaattcg

SEQ ID NO: 11 - DNA, 22 bp, Artificial sequence
  Synthetic sequence
gctagcgtcgactacggagatg

SEQ ID NO: 12 - protein, 5 aa, Artificial sequence
  Synthetic sequence
GGGGS

SEQ ID NO: 13 - protein, 7 aa, Artificial sequence
  Synthetic sequence
RSCVDMV